(19) United States
(12) Patent Application Publication
(10) Pub. No.: US 2026/0334811 A1
(43) Pub. Date: Oct. 29, 2026

(54) TECHNOLOGY FOR INEXPENSIVE AND QUANTIFIED ASSESSMENT OF INFANT SUCKLING BEHAVIOR

(71) Applicant: The Regents of the University of California, Oakland, CA (US)

(72) Inventors: Erin Walsh, San Diego, CA (US); James Friend, San Diego, CA (US); Todd Coleman, San Diego, CA (US); Phuong Truong, San Diego, CA (US); Gopesh Tilvawala, San Diego, CA (US)

(21) Appl. No.: 19/118,940

(22) PCT Filed: Oct. 6, 2023
(86) PCT No.: PCT/US2023/076198

§ 371 Date: Apr. 7, 2025

Related U.S. Application Data
(60) Provisional application No. 63/378,854, filed on Oct. 7, 2022.

(51) Int. Cl. A61B 5/083; A61B 5/00

(52) U.S. Cl. CPC A61B 5/083; A61B 5/682; A61B 5/7267; A61B 5/7271; A61B 5/742; A61B 2503/04

(57) ABSTRACT

Provided herein is a method for evaluating infant suckling behavior, comprising: collecting suckling data from an infant using a vacuum measurement device, transmitting the collected suckling data to a processor, processing the collected suckling data by the processor using a pre-trained Machine Learning (ML) algorithm to identify one or more abnormalities in the infant's suckling behavior, wherein the ML algorithm has been trained on a set of training data comprising a plurality of dimensions associated with the training data, and wherein the identified one or more abnormalities are corresponding to one or more of the plurality of dimensions.

### CROSS-REFERENCE

This application is a national Stage entry of Patent Cooperation Treaty Application No. PCT/US2023/076198 filed Oct. 6, 2023, entitled “TECHNOLOGY FOR INEXPENSIVE AND QUANTIFIED ASSESSMENT OF INFANT SUCKLING BEHAVIOR,” which claims priority to U.S. Provisional Application No. 63/378,854 filed on Oct. 7, 2022, entitled “TECHNOLOGY FOR INEXPENSIVE AND QUANTIFIED ASSESSMENT OF INFANT SUCKLING BEHAVIOR.” The disclosures of which are incorporated herein by reference in their entirety.

### TECHNICAL FIELD

The subject matter described herein relates generally to assessment of infant suckling behavior.

### BACKGROUND

Breastfeeding is a natural process that provides numerous health benefits to both infants and mothers. It is widely recognized that breastfeeding can protect infants from a variety of diseases and conditions, including diabetes, allergies, cardiovascular disease, and other chronic conditions. Mothers who breastfeed also experience health benefits, such as a decreased risk of breast cancer, ovarian cancer, and postpartum depression. Despite these benefits, breastfeeding rates often decline significantly within the first six months after birth. One factor that can contribute to this decline is abnormal infant suckling behavior. Infant suckling is a complex process that involves the coordination of sucking, swallowing, and breathing. Abnormalities in this process can lead to issues such as nipple pain and injury, latch problems, and a decrease in the mother's milk supply. There exist needs to improve monitor and assessment of infant suckling behavior, and subsequent remedial measurements.

### SUMMARY

As discussed, breastfeeding is a natural process that provides numerous health benefits to both infants and mothers, and monitor and assessment of infant suckling behavior may improve breastfeeding process. Infant suckling can be categorized into two types: nutritive sucking and non-nutritive sucking. In nutritive sucking, infants intake fluid from a breast or bottle. In non-nutritive sucking, infants do not receive nutrient flow and the suck is from basic instinct when offered an empty or uninitiated breast, pacifier, finger, or object. Non-nutritive sucking is characterized by suckling vacuum and expression pressure. Various devices and methods have been developed to measure infant non-nutritive suckling. These include catheters, pneumatic and fluid-based instruments, and compact devices. However, these devices and methods have primarily focused on preterm infants, and there has been less consideration of how abnormal non-nutritive suckling shapes could be detected in otherwise healthy full-term infants. Additionally, Machine learning has been applied in various fields, including healthcare, to analyze complex data and detect patterns or anomalies. In the context of infant suckling, machine learning may be used to rapidly interpret infant suckling measurements and detect abnormalities.

Methods, systems, and articles of manufacture, including computer program products, are provided for intelligent generation of unit tests. In one aspect, there is provided a method for evaluating infant suckling behavior, comprising: collecting suckling data from an infant using a vacuum measurement device, transmitting the collected suckling data to at least one processor, processing the collected suckling data by the at least one processor using a pre-trained Machine Learning (ML) algorithm to identify one or more abnormalities in the infant's suckling behavior, wherein the ML algorithm has been trained on a set of training data comprising a plurality of dimensions associated with the training data, and wherein the identified one or more abnormalities are corresponding to one or more of the plurality of dimensions.

In some variations, one or more of the features disclosed herein including the following features can optionally be included in any feasible combination. In some variations, the vacuum measurement device comprises a pacifier integrated with a feeding tube. In some variations, the method further comprising providing on a display a real-time visualization of the collected suckling data. In some variations, the method further comprising providing, on the display, a real-time visualization of the collected suckling data in comparison to a population mean and standard deviation. In some variations, the method further comprising providing, on the display, a graphical representation of the identified one or more abnormalities. In some variations, the plurality of dimensions comprise mean suck vacuum, maximum suck vacuum, suckling frequency, burst duration, sucks per burst, and vacuum signal shape. In some variations, the ML algorithm is configured to apply an unsupervised anomaly detection method using the Mahalanobis distance to detect abnormalities in the collected suckling data.

In one aspect, there is provided a system comprising a vacuum measurement device configured to collect suckling data from an infant; and at least one processor configured to receive the collected suckling data and process the collected suckling data using a pre-trained Machine Learning (ML) algorithm to identify one or more abnormalities in the infant's suckling behavior, wherein the ML algorithm has been trained on a set of training data comprising a plurality of dimensions associated with the training data, and wherein the identified one or more abnormalities are corresponding to one or more of the plurality of dimensions.

In some variations, one or more of the features disclosed herein including the following features can optionally be included in any feasible combination. In some variations, the vacuum measurement device comprises a pacifier integrated with a feeding tube. In some variations, the vacuum measurement device comprises a pacifier, a feeding tube, wherein a first end of the feeding tube is coupled with the pacifier, and a pressure sensor that is connected with a second end of the feeding tube, wherein the pressure sensor is configured to collect the suckling data from the infant. In some variations, the system further comprises a display configured to provide real-time visualization of the collected suckling data. In some variations, the display is further configured to provide a real-time visualization of the collected suckling data in comparison to a population mean and standard deviation. In some variations, the display is further configured to provide a graphical representation of the identified one or more abnormalities. In some variations, the plurality of dimensions comprise mean suck vacuum, maximum suck vacuum, suckling frequency, burst duration, sucks per burst, and vacuum signal shape. In some variations, the ML algorithm is configured to apply an unsupervised anomaly detection method using the Mahalanobis distance to detect abnormalities in the collected suckling data.

In another aspect, there is provided a vacuum measurement device, comprising: a pacifier, a feeding tube with a first end of the feeding tube coupled with the pacifier, and a pressure sensor that is connected with a second end of the feeding tube, wherein the pressure sensor is configured to collect suckling data from an infant. In some variations, one or more of the features disclosed herein including the following features can optionally be included in any feasible combination. In some variations, the feeding tube is shorter than 48 cm. In some variations, the vacuum measurement device further comprises a transmission unit configured to transmit the collected sucking data to at least one processor. In some variations, the pressure sensor is configured to detect and measure changes in pressure within the pacifier as the infant suckles. In some variations, the pressure sensor is configured to detect and measure a plurality of dimensions comprising mean suck vacuum, maximum suck vacuum, suckling frequency, burst duration, sucks per burst, and vacuum signal shape.

In some example embodiments, there may be provided a non-nutritive suckling (NNS) system to measure and analyze intraoral vacuum of full-term neonates in real-time as substantially described and shown herein.

The details of one or more variations of the subject matter described herein are set forth in the accompanying drawings and the description below. Other features and advantages of the subject matter described herein will be apparent from the description and drawings, and from the claims.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a block graph illustrating a system for evaluating infant suckling behavior, according to one or more embodiments of the present disclosure;

FIG. 2 depicts a typical intraoral vacuum labelled with characterization parameters, according to one or more embodiments of the present disclosure;

FIG. 3 depicts a suckling signal generated by an infant utilizing the system depicted herein, according to one or more embodiments of the present disclosure;

FIG. 4 depicts signals representative of the three distinguishable profiles found in NNS signal of the thirty infants tested, according to one or more embodiments of the present disclosure;

FIGS. 5(a)-(e) graphically illustrates the statistical differences between each group based on their characteristics, according to one or more embodiments of the present disclosure;

FIGS. 6(a)-(e) graphically illustrates the statistical differences between each group based on their characteristics, according to one or more embodiments of the present disclosure;

FIG. 7 depicts a block diagram depicting an example system 700 comprising a client-server architecture and network configured to perform the various methods described herein, according to one or more embodiments of the present disclosure;

FIG. 8 depicts an example flowchart for a process 800 for evaluating infant suckling behavior, according to one or more embodiments of the present disclosure;

FIG. 9 depicts signals representative of a 60-second non-nutritive suckling shape as measured, according to one or more embodiments of the present disclosure.

FIG. 10 depicts a visualization of a typical infant's NNS behavior, according to one or more embodiments of the present disclosure.

FIG. 11 depicts a visualization of the non-nutritive suckling (NNS) behavior of an infant characterized by prolonged bursts of suckling activity, according to one or more embodiments of the present disclosure.

FIG. 12 depicts a visualization of the non-nutritive suckling (NNS) patterns of an infant who exhibits notably weak and sporadic suckling episodes, according to one or more embodiments of the present disclosure.

FIG. 13 illustrates a plot comparing the Robust distance and the Mahalanobis distance derived from the NNS data of all 91 subjects, according to one or more embodiments of the present disclosure.

FIG. 14 illustrates presents a plot comparing the Robust distance and the Mahalanobis distance for the NNS data across the 91 infants, according to one or more embodiments of the present disclosure.

FIG. 15 provides a comprehensive visualization of the impact of a frenotomy on subject 71, specifically focusing on the non-nutritive suckling (NNS) parameters, according to one or more embodiments of the present disclosure.

FIG. 16 provides the discernible influence of a frenotomy on subject 60, particularly emphasizing the non-nutritive suckling (NNS) metrics, according to one or more embodiments of the present disclosure.

FIG. 17 presents a comparative analysis of subject 22's non-nutritive suckling (NNS) metrics pre- and post-frenotomy, according to one or more embodiments of the present disclosure.

FIG. 18 provides a comparative visualization using the Robust distance versus Mahalanobis distance plot, specifically focusing on the NNS data from cases where a frenotomy was conducted, according to one or more embodiments of the present disclosure.

### DETAILED DESCRIPTION

Breastfeeding is a natural biologic function that fosters attachment and safeguards the health of mothers and babies. By breastfeeding, mothers experience lower risks of reproductive organ cancers, type II diabetes, cardiovascular disease and mental health disorders, while infants experience lower risks of infectious diseases, gastrointestinal and respiratory health issues, allergies, type II diabetes, hypertension, and obesity. While the advantages of breast milk far outweigh formula, the rate of exclusive breastfeeding at six months post-birth plummets to only 25%, according to the CDC's 2018-2019 National Immunization Survey.

Early breastfeeding diagnostics to identify poor latch and suck are essential for timely interventions and support for the mother and infant to help them avoid resorting to formula. Presently, feeding clinicians and pediatricians assist mothers and infants with breastfeeding challenges, yet are constrained by the absence of tools to objectively quantify suck vacuum, a key aspect of successful breastfeeding. Existing assessment methods are essentially qualitative measurements, such as digital suck assessment using a gloved finger to determine infant suckling vacuum. While more elaborate assessment scales do exist, few clinicians are trained to administer and interpret them. Due to this, both objectivity and consensus among the clinical community are lacking, leaving the diagnosis of breastfeeding difficulties in an ambiguous limbo and leading to a variety of interventions that may be unwarranted (e.g. frenotomy).

In recent years, several devices and systems have been developed to quantify the suckling profile and oral-motor coordination of premature infants. These systems principally address the challenge of oral feeding readiness in premature infants by measuring their intraoral suction (vacuum) and expression (contact) pressure. While not posed to diagnose breastfeeding problems in full-term infants, some of these systems show promise in doing so. Grassi, et al., for example, developed a sensorized pacifier that measures suction and expression pressures using two integrated pressure transducers, displaying measurement results via a simple graphical user interface (GUI). Lau, et al., studied pressure measurements from two sensorized catheters attached to a gloved index finger. Ebrahimi, et al., devised a portable compact intraoral pressure measurement system that includes features such as a custom printed circuit board, wireless communication, and a rechargeable battery. The FDA-approved NTrainer by Capilouto, et al., measures the displacement of the tongue (expression pressure) and incorporates pneumatic actuation to help facilitate infant oromotor skills. Geddes et al., utilizes ultrasound along with pressure transducers to correlate vacuum characteristics to milk intake during nutritive sucking. These devices, along with many others proposed in the literature, all reflect an effort to provide objective quantification of infant intraoral vacuum. However, even with all these capable devices, there remains a challenge in devising a clinically feasible system. Namely, none of these systems consider most factors important in clinical use, such as sterilization, real-time analysis with immediate feedback to the clinician, ease of use, measurement accuracy and repeatability, and variability in infant suckling preferences. These factors directly impact the translational aspects and usability of the technology in clinic and are critical to consider in the engineering design process.

Disclosed herein there is disclosed a non-nutritive suckling (NNS) system to measure and analyze intraoral vacuum of full-term neonates in real-time. The disclosed system considers the feasibility of clinical use and provides an objective alternative to the standard digital suck assessment. Specifically, measurements are performed (using full term infants) on the suckling vacuum to extract the following objective parameters: the mean and maximum vacuum amplitude, suckling frequency, number of suckling events per burst, burst duration, and number of bursts per minute. Findings show that the infants' intraoral profile produce distinctive vacuum responses that can in turn be used to identify orofacial issues. These signals can be categorized and provide a framework for studying and oromotor dysfunctions in future studies.

To develop a robust system that is feasible for clinical use, the disclosed system design approach for the NNS system considers its utilization and interaction with both clinicians and infants. Parameters of the sensing system, described in Table I, and the configuration of the components were considered as a part of the design of the NNS system to ensure clinical feasibility. Table I also summarizes the design requirements for the proposed system based on the advantages and drawbacks of existing systems reported in the literature.

### Example of NNS System Hardware

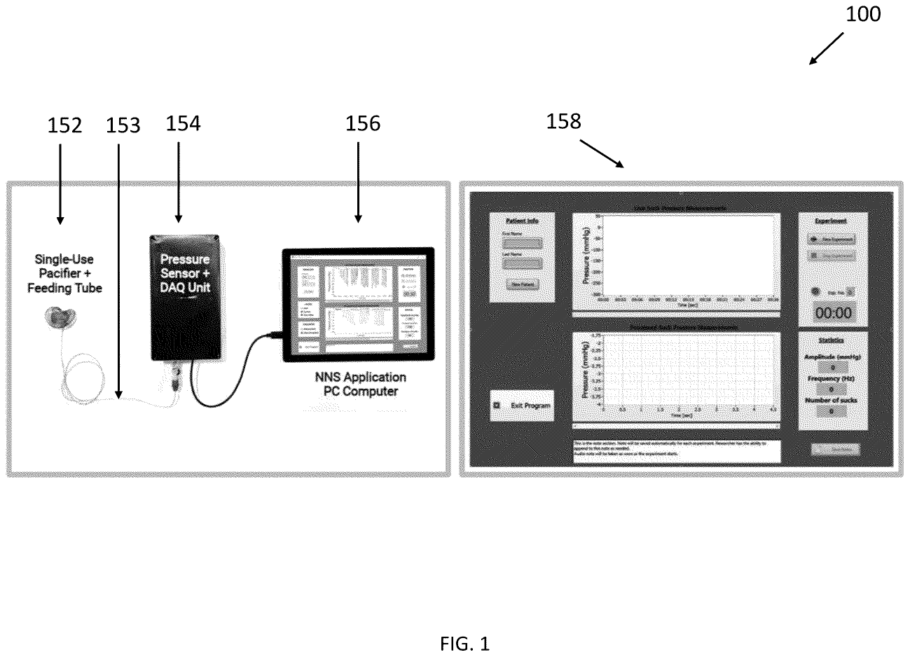

To achieve these design requirements, an example of an NS system as shown in FIG. 1 is implemented that may be comprised of several main components: a single-use modified pacifier, a pressure sensor, an optional data acquisition unit, and a custom-made software interface as shown at FIG. 1. This configuration takes into account the safety implications of including the pressure sensor in the pacifier with wired connections, the issues with wireless communications potentially being unsecure and unacceptable in light of patient privacy regulations (e.g., HIPAA, the Health Insurance Portability and Accountability Act of 1996), and the need to provide real-time data via a dedicated computer to the clinician.

TABLE I
DESIGN PARAMETERS CONSIDERED IN THE NNS SYSTEM
TO MEET CLINICAL AND ENGINEERING REQUIREMENTS
NEEDED FOR CLINICAL FEASIBILITY.

Design Requirements

| | |
|---|---|
| Ease of use | Hardware and software must be intuitive for clinicians to use and |
| | manage with minimal training |
| Biosafety | Components in direct contact with saliva, bodily fluids and oral |
| | cavity must be sterilized before use and must be sterilized or |
| | disposed after each use |
| Adaptability | Infant pacifier preferences may vary; suckling unit must be |
| | versatile in adapting to various pacifier types |
| Biocompatibility | Components interfacing with infant must meet biocompatibility |
| | safety requirements |
| Electrical Safety | Electrical components must operate within International |
| | Electrotechnical Commission (IEC) safety limits |
| Accuracy | Pressure sensing unit dynamic range must be able to measure |
| | physiological range of intraoral vacuum of infants (0 mmHg |
| | to −400 mmHg) |
| Repeatability | System measurements must be repeatable as needed to track infant |
| | vacuum over time |

For example, the pacifier component was fabricated using a commercial teat (Orthodontic Pacifier, NUK) integrated with a 36-inch non-collapsible feeding tube (Kangaroo Neonatal & Pediatric Feeding Tube, Covidien). While this teat was selected for its shape and fit with the infants' oral anatomy, the modularity of the system permits quick substitution with any pacifier shape and type preferred by the infant. To integrate the feeding tube with pacifier, a 1 mm diameter biopsy punch was used to create an opening at the tip of the pacifier and the feeding tube was passed through the opening. Next, 0.8 mL of polydimethylsiloxane (Sylgard 184, Dow Corning), a biocompatible and inert non-toxic silicone was poured into the inner cavity of the pacifier to hold the feeding tube in position. The silicone was cured in a 40\hat{ }°C oven for 24 hours. Once integrated, the modified pacifier was cleaned with water and mild soap and dried. The unit was bagged and sterilized under 275 nm ultraviolet light (Sterilizer and Dryer, VANELC) for 35 minutes. The bio-compatibility and safety of the modified pacifier was considered in the design. We limit infant exposure to any unknown materials and only consider those that are accepted or widely used. A silicone pacifier (commercially available) integrated with a medical-grade PVC feeding tube are the only materials in contact with the infant, ensuring biocompatibility and safety. In circumstances where the infant rejects the pacifier or has a known allergic reaction to the pacifier material, the pacifier can be substituted for any preferred pacifier such as the Soothie (Philips AVENT), a standard pacifier used in hospitals.

For example, a piezoresistive pressure sensor (MPX5100AP, NXP Semiconductors) was selected with an operating range of 110-860 mmHg (absolute) to fit the application and system design requirements of neonatal suckling dynamic range. Intraoral vacuum of typical neonates during suckling has been reported to be 375-825 mmHg. To acquire intraoral vacuum measurements, the pressure sensor was coupled with a data acquisition board (myDAQ, National Instruments) and connected to a computer with a graphical software interface (LabVIEW, National Instruments) for simple analysis and data visualization by the clinician. The sampling frequency is set to 1000 Hz to sample at a greater rate than the suckling frequency, which is reported in the literature to be within the 1.5 Hz to 2.5 Hz range. The maximum output voltage in the device is 5 VDC, well below the standard limit for contact with a human (~30 VDC). Moreover, the maximum current available in the device is about 2 mA. These aspects make the device intrinsically safe according to IEC standards 61140, 60364, 61010-1, and 60479. The data acquisition board and sensor are entirely contained in an insulated box without possibility of making contact with the infant.

For example, the pacifier and feeding tube unit connects to the pressure sensor through a quick connect luer lock allowing for ease of use. The design considers the clinical workflow: To use the unit, a clinician would (1) connect the hardware to a computer via USB, (2) open the NNS software, (3) open a new pacifier unit, (4) connect the tubing to use, and (5) press start experiment to collect data. All of this can be done in less than a minute with minimal training required. Finally, since the components are relatively low cost, the system is designed such that the pacifier-feeding tube unit is single-use (disposable) to minimize both cross contamination of fluids such as saliva between patients and the need to clean or sterilize the device after each measurement. The disposability and quick connect/disconnect design features helps streamline the integration of the device into the fast-paced clinical workflow and allows the clinician to quickly test patients as a part of their routine examination schedules.

### Example of System Software Design and Signal Processing

In an example implementation, the NNS system software is designed to record intraoral vacuum measurements in real time and immediately process and display for the clinician to see while the data collection is underway. This allows clinicians to utilize information for rapid diagnosis and dynamically adjust to retake measurements as needed. Table 2 summarizes the key software features that enable rapid diagnosis in a clinical setting.

TABLE II
SOFTWARE FEATURES AND CAPABILITIES OF THE NNS
APPLICATION. ITS DESIGN FOCUSES ON THE CLINICAL
NEEDS OF THE MEDICAL PROFESSIONAL IN A CLINICAL
BREASTFEEDING ASSISTANCE SETTING.

| | |
|---|---|
| Real-Time Data | Vacuum measurements are collected and shown on the computer |
| | screen in real-time as the pacifier is used by the patient. |
| | Clinicians can adjust and continue measurements, end the |
| | experiment, or restart measurements for the same patient. |
| Immediate Analysis | Once measurements are completed, the software algorithm will |
| | automatically compute the characterization parameters such as |
| | the max amplitude, frequency, number of sucks, and burst |
| | duration for the entire profile. |
| ROI Analysis | Clinicians can utilize the interface to segment the data for |
| | analysis in specific regions of interest (ROI) of the vacuum |
| | profile. The characterization parameters are automatically |
| | recalculated and displayed. |
| Note Taking | Audio recording is automatically started for clinicians to record |
| | any verbal notes during testing. Written notes are also featured |
| | and automatically saved with raw data files corresponding to the |
| | patient. |

The NNS application was designed and built using LabVIEW, a graphical programming environment, although other types of software packages may be used as well. The custom program was packaged into an executable application that can be deployed on any PC that is readily available in the clinic without the need of the native LabVIEW software. This allows for ease of adoption and reduces barriers to entry. The NNS app was designed with an intuitive user interface where the clinicians can enter patient information, start (or stop) experiments, and view the pressure profile and key metrics in real time. Clinicians may also magnify regions of interest for closer inspection and analysis of the shape of the suckling signal.

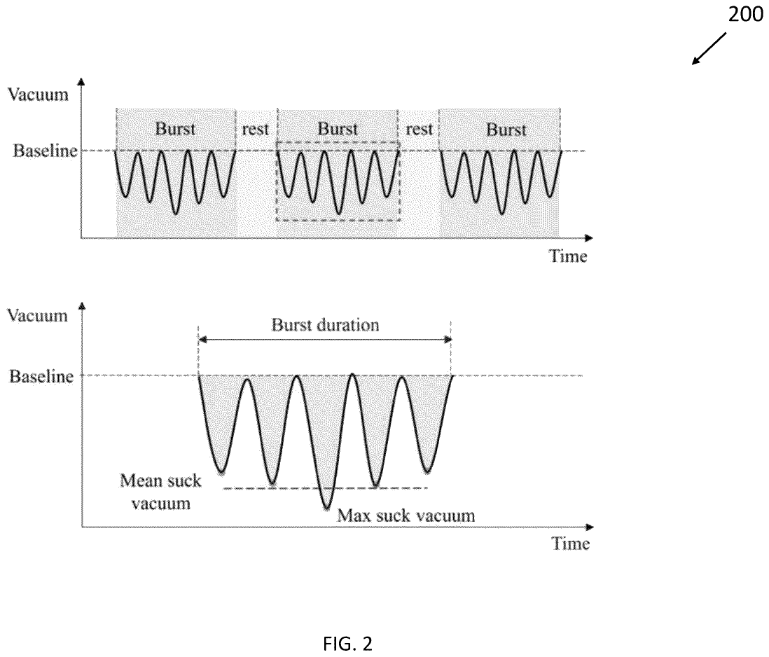

Once the signal is acquired, characterization is performed automatically by the software. Table III describes the parameters extracted from the NNS signal. The analysis sequence of the app begins with the detection of peaks and valleys for the full suckling profile. Referring to FIG. 2, one suck cycle is defined as having a minimum suck amplitude of 10 mmHg based on reported definitions in the literature. These values are used to establish the threshold of the peak-valley detection algorithm. A burst is defined as two or more consecutive suck cycles with a minimum rest period of one second between bursts. From the NNS profile, other characteristics can be extracted. The suck amplitude is defined by the average measured amplitude of the infant's vacuum placed upon the pacifier over the trial. The time period between two successive valleys of locally maximum suck vacuum are collected over all the suckling events and used to calculate the average suck frequency, both for each burst and for the entire trial.

TABLE III
THE FEATURES EXTRACTED FROM THE SUCKLING
SIGNAL BY THE NNS SOFTWARE AND WHICH HELP
TO CHARACTERIZE THE INFANT'S SUCKLING.

| | |
|---|---|
| Mean Suck Vacuum | Average amplitude within ROI |
| Max Suck Vacuum | Maximum amplitude within ROI |
| Frequency | Number of sucks per second |
| Burst Duration | Duration of a cluster of sucks between rests |
| Bursts per Minute | Average number of clustered sucks per minute |
| Sucks per Minute | Average number of sucks per minute |
| Sucks per Burst | Average number of sucks across all burst events within one |
| | recording session |

And, for example, an interactive cursor allows the clinician to extract these features for a specific region of interest (ROI). The application automatically updates values as the clinician selects different ranges of the measured data via the GUI. This enables the clinician to focus on specific time points or ROIs in the suck profile for a closer analysis.

### Examples of Clinical Testing and Protocol

Thirty healthy term newborns (gestational age: 37-42 weeks) under 30 days of age were recruited from both the UC San Diego Health Department of Otolaryngology's Center for Voice and Swallowing and the Pediatrics Department. The infant inclusion criteria for the study were: (1) infants 4-30 days old (critical period to establish breastfeeding); (2) healthy; no significant birth or post-partum complications; and (3) no known allergy to silicone or elastomers typically used for pacifiers and bottle nipples. The study aimed to measure infant suckling vacuum using the NNS system to establish a norm of sucking signal characteristics. Testing occurred during a lactation consultation visit (Center for Voice and Swallowing) and during outpatient pediatric visits (UC San Diego General Pediatrics). Approval from the Institutional Review Board at UC San Diego (IRB 800070 approved 13 Sep. 2021) was obtained before recruitment started. Parents were informed of the nature of the study and consented before the experiment began. Infants underwent a routine weight and physical exam. After an initial routine evaluation, infants were offered the NNS system pacifier. Intraoral vacuum was recorded for a duration of 60 seconds.

### Example Results

FIG. 1 depicts a block graph illustrating a system 100 for evaluating infant suckling behavior, according to one or more embodiments of the present disclosure. As show in FIG. 1, the system 100 may comprise a pacifier 152, a feeding tube 153, a pressure sensor 154, an optional data acquisition board (DAQ) unit, a NNS application interface 156, and/or a display 108. In some embodiments, as shown in FIG. 1, one end of the feeding tube 153 may be coupled with the pacifier, and the other end of the feeding tube 153 may be connected with a pressure sensor 154. The pressure sensor 154 may collect data such as pressure variations during suckling, which may be indicative of the infant's oral motor behavior. The data collected by the pressure sensor 154 may be transmitted to the DAQ unit, if present, for initial processing. The feeding tube 153 may have a length ranging from 36 to 48 cm. In some embodiments, the feeding tube 153 may have a length less than 48 cm, less than 45 cm, less than 42 cm, less than 40 cm, less than 35 cm, less than 30 cm, less than 25 cm, less than 20 cm, less than 15 cm, etc. This length is optimized to ensure accurate and timely registration of the vacuum created by the baby's sucking action. If the tube were longer than an optimal length (e.g., this optimal length may be pre-determined based on, for example, a diameter of the feeding tube 153), it could potentially introduce delays and errors in the signal due to the increased time it would take for the vacuum to be registered by the recording device.

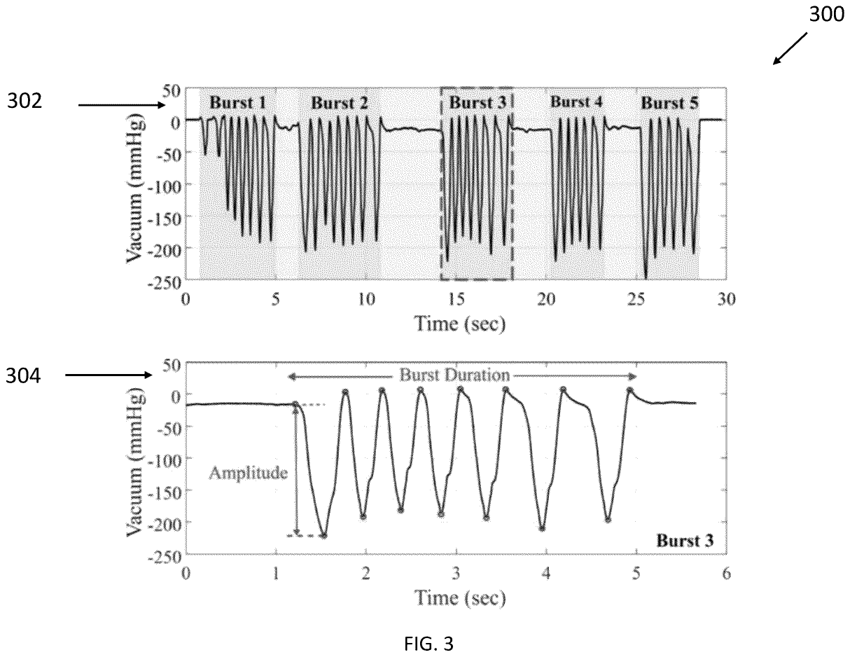

In some embodiments, the optional DAQ unit may be configured to digitize the analog pressure data for further analysis. In some embodiments, the DAQ unit may be coupled to the NNS application interface 156, which may further process the digitized pressure data to extract relevant suckling metrics such as suckling pressure, frequency, duration, and/or suckling burst patterns. The NNS application interface 156 may use one or more pre-determined algorithms for analyzing the digitized pressure data and identifying any abnormalities or variations in the infant's suckling behavior. In some embodiments, the system 100 may further include a display 108, which may be coupled to the NNS application interface 156. The display 158 may be configured to present the analyzed suckling metrics and/or any identified abnormalities to a user, such as a healthcare provider or a parent. The display 158 may include graphical representations, numerical values, and/or textual descriptions of the analyzed suckling metrics, and may provide an intuitive interface for the user to interact with the system 100. In some embodiments, the system 100 may be configured to provide real-time monitoring and analysis of the infant's suckling behavior. The NNS application interface 156 may include one or more real-time processing algorithms to evaluate the infant's suckling behavior as it occurs. The real-time monitoring and analysis may be beneficial for identifying any immediate issues or concerns with the infant's suckling behavior, and may provide instant feedback to the user. Additionally or alternatively, the system 100 may be configured for wireless communication with other devices or systems, such as a computer or a mobile device, via one or more communication interfaces (not shown). The wireless communication may allow for remote monitoring and analysis of the infant's suckling behavior, and may facilitate data sharing with other healthcare providers or researchers for further evaluation or study. In some embodiments, the pacifier 152, feeding tube 153, and pressure sensor 154 may be fabricated as a single integrated unit to simplify the design and enhance the durability and hygiene of the system 100. The integrated unit may also minimize the number of components that need to be separately cleaned and sterilized, which may be beneficial in a healthcare or research setting. The results from the clinical study validate the ability of the NNS system to measure intraoral vacuum. Clinicians utilized the system with minimal training and were able to incorporate the system into their workflow. The characteristics described in Table III were collected over 60 seconds. FIG. 2 depicts a typical intraoral vacuum labelled with characterization parameters, according to one or more embodiments of the present disclosure. FIG. 3 depicts a suckling signal generated by an infant utilizing the system depicted herein, according to one or more embodiments of the present disclosure. FIG. 3 may comprise a representative snapshot of a typical infant intraoral vacuum profile, including the details of a particular burst event. As shown in FIG. 3, element 304 may illustrate details of a region of interest (i.e., burst 3 shown in element 302) generated by the NNS software 106, so to provide real-time analysis of the signal within the region of interest defined by one or more users. The analysis and visualization contains peak and valley detection to characterize the signal parameters such as burse duration, maximum suck vacuum, and mean suck vacuum. Table IV summarizes the parameters extracted from the cohort of 30 infants' suckling data. Values extracted are consistent with those previously reported in the literature as shown in the table, demonstrating the systems' ability to capture intraoral vacuum over time. Upon closer inspection of the suckling signals by magnifying a suckling burst, subtle differences are observed in the vacuum transducer's signal.

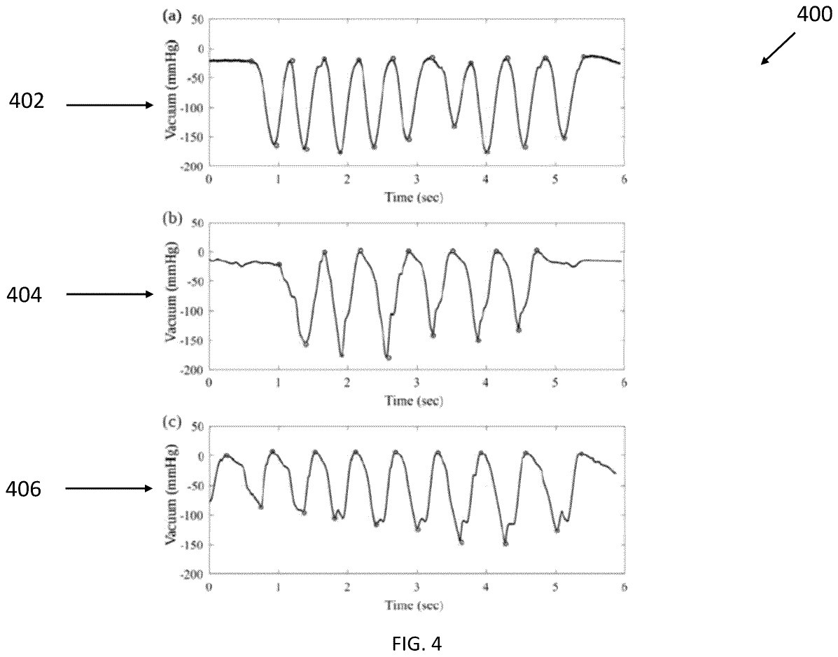

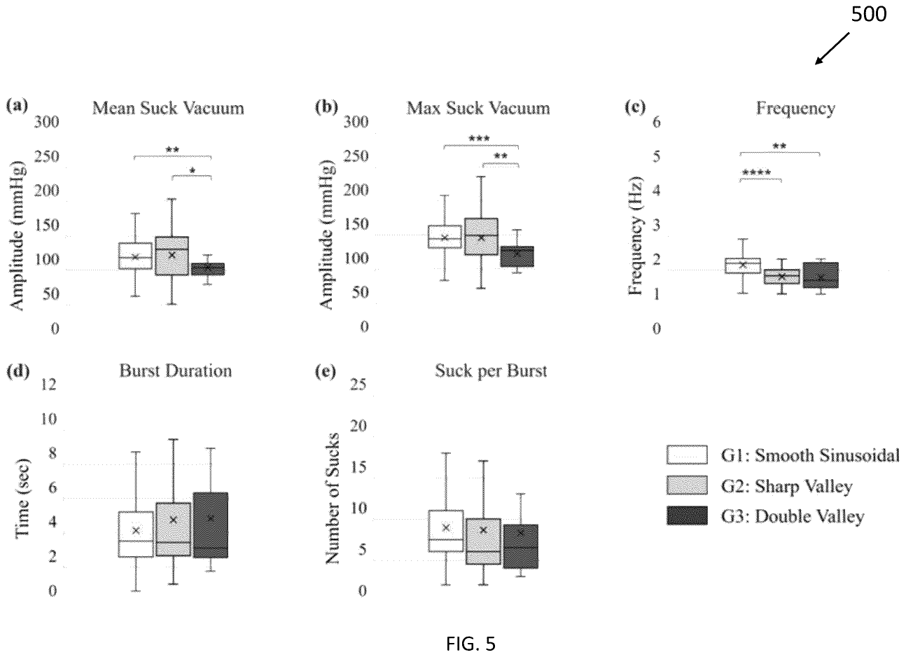

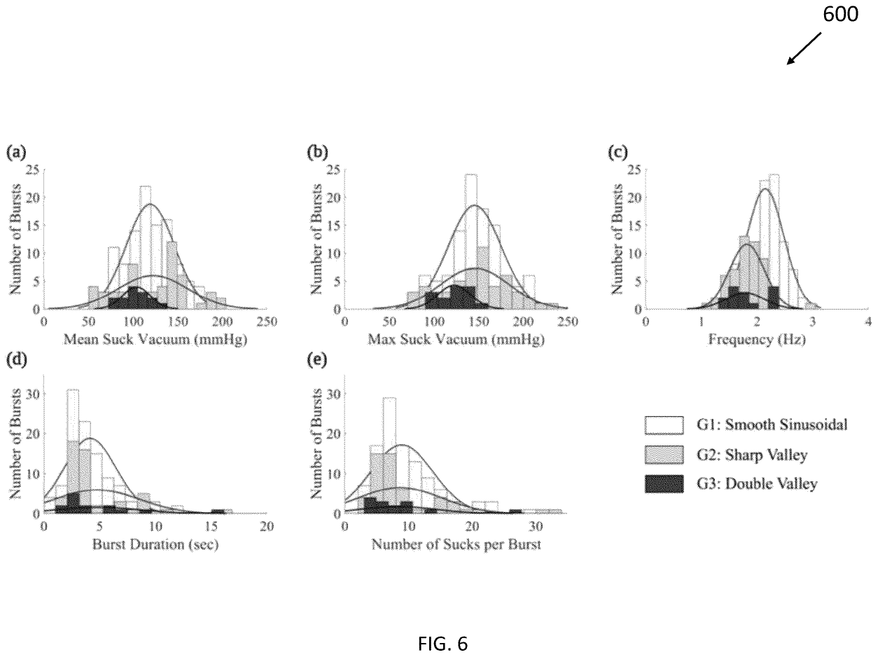

FIG. 4 shows signals representative of the three distinguishable profiles found in NNS signal of the thirty infants tested. The three shapes are classified as: smooth sinusoidal 402, sharp valley 404, and double valley 406. While the factors contributing to these varying shapes are not yet known, the cohort of infant profiles are grouped into three groups corresponding each of the three shapes. FIGS. 5(a)-(e) and FIGS. 6(a)-(e) graphically illustrate the statistical differences between each group based on their characteristics.

In a statistical analysis, classified the shape of the profiles into three main categories: smooth sinusoidal (18 neonates, 106 bursts), sharp valley (10 neonates, 53 bursts), double-valley (2 neonates, 14 bursts). Histograms of the NNS parameters from the three groups are shown in FIGS. 6(a)-(e). It can be observed in FIGS. 6(a)-(c) that the distributions of mean suck vacuum, max suck vacuum and frequency are normally distributed. This was confirmed using the Shapiro-Wilk normality test. A 2-sample Welch's t-tests, which requires that data to be normally distributed, was performed on these parameters across the three groups to find any statistical differences between the groups. The results are shown in Table V. There were several statistically significant differences in mean suck vacuum, max suck vacuum and frequency between the three groups. There were no significant differences observed in burst duration and number of sucks per burst. These profile characteristics persist throughout the entire suckling signal of each infant. If the infant displays a signal shape corresponding to a sharp valley, this pattern can be observed throughout the entire suckling profile.

TABLE IV
SUMMARY OF RESULTS TABLE COMPARING EXTRACTED PARAMETER VALUES
COLLECTED IN THIS STUDY AND THOSE REPORTED IN THE LITERATURE.

| Parameters | This Work | Grassi | Zimmerman | Ebrahimi |
|---|---|---|---|---|
| Number of Subjects | 30 | 9 | 16 | 4 |
| Mean Suck Vacuum (mmHg) | 118.6 (30.8) | — | 64.6 (22.9) | — |
| Max Suck Vacuum (mmHg) | 143.6 (32.2) | 164.1 (38.6) | — | 225.5 (62.9) |
| Frequency (Hz) | 2.01 (0.37) | — | 2.16 (0.35) | 2.40 (0.52) |
| Sucks per Burst | 8.8 (5.5) | 6.9 (1.0) | 5.6 (3.1) | 5.9 (0.6) |
| Burst Duration (sec) | 4.4 (3.0) | 2.9 (0.6) | 2.5 (1.4) | 2.6 (0.2) |
| Sucks per Minute | 70.7 (16.9) | — | 28.1 (25.6) | 60.5 (10.0) |
| Bursts per Minute | 7.2 (3.1) | 9.3 (2.1) | 4.1 (2.7) | 10.3 (1.0) |

TABLE V
Welch's t-test results comparing mean suck vacuum, max suck vacuum
and frequency between the three groups of NNS profile shapes.

| Parameters | Groups | t | p |
|---|---|---|---|
| Mean Suck Vacuum (mmHg) | 1 vs. 2 | −0.49 | 0.62 |
| | 1 vs. 3 | 3.16 | 0.005 |
| | 2 vs. 3 | 2.72 | 0.010 |
| | 1 vs. 2 | −0.02 | 0.99 |
| Max Suck Vacuum (mmHg) | 1 vs. 3 | 4.04 | <0.001 |
| | 2 vs. 3 | 3.22 | 0.003 |
| | 1 vs. 2 | 6.15 | <0.001 |
| Frequency (Hz) | 1 vs. 3 | 3.70 | 0.002 |
| | 2 vs. 3 | 0.33 | 0.74 |

* Bold values are statistically significant.

The results from this study show measurements in agreement with values reported in the literature. Our system demonstrates features and capabilities that addresses the clinical needs of an easy-to-use, accurate, and safe system. The immediate feedback of suckling performance allows clinicians to troubleshoot breastfeeding problems with greater accuracy using objective data.

Our region of interest analysis show differences in profile shapes which may be further investigated as it may relate to infant oral motor restrictions, and other characteristics related to oral motor function. A detailed burst analysis of the NNS data from the 30 neonates showed that there were statistically significant differences in key NNS parameters between neonates with different suckling profile shapes. These results suggest that the shape of the suckling profile can play an important role in evaluating the suckling mechanics of the infants. As more data from a larger population of neonates becomes available, there may provide further investigation into the shape of the infant suckling profile as it relates to oral motor functions or disorders and also map key parameters of the profile, e.g., the sharpness of a given suckling vacuum event, to the severity of certain conditions.

The technical improvements that can be implemented in such a system include reducing the size of the data acquisition unit and incorporating Bluetooth capabilities to eliminate the cable to the computer. While this may further reduce the size of the system, it may also increase the system's operating complexity due to wireless pairing, data security considerations, and biosafety challenges caused by the proximity of the suckling unit to the electronics.

Clinical testing in this study examines a small sample size of infants and will expand further to investigate the system's ability to capture profiles that reflect poor vacuum, coordination, fatigue, respiratory asynchrony, and varying maturation levels. More importantly, we aim to further investigate the shape of the infant suckling profile as it relates to oral motor functions or disorders. We hypothesize that existing systems have not yet demonstrated the subtle changes in the signal due to engineering design problems such as the use of large elastic tubing and the presences of large dead air volumes within the system that may dampen or reduce the sensitivity of the measurements.

Expression pressure is a common measurement capability of systems in the literature aimed at tracking premature infant oral motor feeding readiness. This typically occurs in bottle feeding. Our aim is to target breastfeeding, therefore, future iterations of the system may be modified for an infant's suckling assessment at the breast. The pacifier can be removed and affixed to feeding tubes placed in the mouth while the baby is nursing. This permits comparison of non-nutritive and nutritive suckling skills. Such a system is reported in the literature by Chen et al. and can be further investigated through larger studies with more infants in various clinical environments to better determine the feasibility of the feeding-tube system.

In this paper, we report on the design of a non-nutritive suckling system. We demonstrated use and application of the system in a clinical environment: a specialist clinic and a general pediatric facility. Thirty neonates were enrolled in the study and their non-nutritive suckling profile was successfully recorded and analyzed in real time. The proposed system allows for objective measurements and quantitative analysis of an infant's suckling profile. The system software interface automatically extracts features from the profile including the maximum and mean vacuum amplitude, suckling frequency, mean suck cycle, number of sucks, number of bursts, and the burst duration.

Like with all available systems and devices in the literature, the broader adoption of this technology in routine clinical practice will be a key challenge. Our future work will investigate the interpretation of these signals with respect to the norm (e.g., burst duration as it relates to endurance, maximum amplitude as it relates to suck vigor, etc.). As we collect more infant suckling profiles, this will enable us to establish a clear understanding of normal versus abnormal patterns of suckling, perhaps correlated to specific medical conditions at first identified by other means. These subtle suckling deviations can better distinguish infant-based interventions to optimize breast milk intake.

Ultimately, the challenge of diagnosing breastfeeding issues in mother-infant dyads remains a very complex and multidimensional problem. The system aims to remove a facet of subjectivity in digital suckling examinations to provide objective quantification of suckling and work towards clinical consensus within the medical and clinical community. This is with the overall goal of helping infants and mothers reach positive breastfeeding outcomes through referral and intervention pathways based on objective measurements. Extended applications of this system can include research of oral-motor or neurological development in infants, at-home intraoral vacuum monitoring system for infants, and as a rapid diagnostics tool in hospitals.

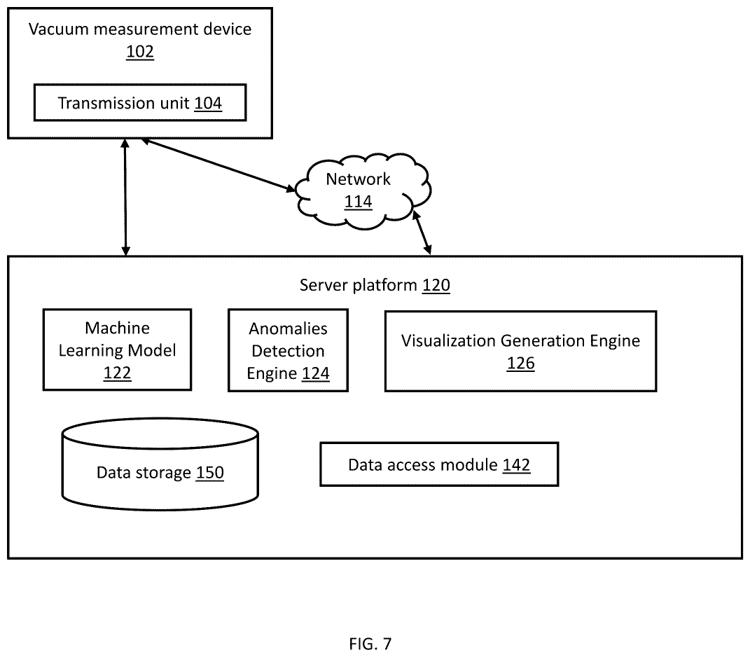

FIG. 7 is block diagram depicting an example system 700 comprising a client-server architecture and network configured to perform the various methods described herein. A platform (e.g., machines and software, possibly interoperating via a series of network connections, protocols, application-level interfaces, and so on), in the form of a server platform 120, provides server-side functionality via a communication network 114 (e.g., the Internet or other types of wide-area networks (WANs), such as wireless networks or private networks with additional security appropriate to tasks performed by a user) to one or more vacuum measurement device 102.

In at least some examples, the server platform 120 may be one or more computing devices or systems, storage devices, and other components that include, or facilitate the operation of, various execution modules depicted in FIG. 7. These modules may include, for example, a machine learning (ML) model 122, an anomaly detection engine 124, a visualization generation engine 126, a data access module 142, and a data storage 150. Each of these modules is described in greater detail below.

In some embodiments, the ML model 122 may be unsupervised models, distance-based models, probabilistic models, ensemble-based methods, etc. For example, unsupervised training for the model 122 may be utilized to generate a ML model that may capture the abnormalities in suckling data. In some embodiments, distance-based models, such as Mahalanobis Distance and/or k-Nearest Neighbors (k-NN) may be utilized. For example, a Mahalanobis Distance model, which takes into account the covariance among variables, can be utilized to identify anomalies. In some embodiments, the model may be an Isolation Forest model, which is an ensemble-based model that isolates anomalies rather than normal instances, making it efficient for anomaly detection in high-dimensional datasets.

The ML model 122 may be trained on a training dataset, wherein the training dataset may comprise a plurality of dimensions associated with the training data. For example, the dimensions may comprise mean suck vacuum, maximum suck vacuum, suckling frequency, burst duration, sucks per burst, and vacuum signal shape. The training process for the ML model 122 starts with data preprocessing which may comprise noise reduction and normalization. For instance, normalization may ensure that all data dimensions have equal or assigned weight, which can be important for distance-based models like Mahalanobis Distance.

Once preprocessing is completed, the cleaned and structured data is fed into the ML model 122. In some embodiments, the model 122 may be subjected to a feature selection or extraction phase, where the most relevant attributes are chosen or transformed versions of them are created to represent the underlying patterns more effectively. During the training phase, the model 122 may learn the underlying patterns and relationships between different dimensions in the dataset. For unsupervised learning models, this means understanding the structure and distribution of normal suckling behaviors. Iterative optimization techniques, like gradient descent, may be utilized to refine the model's parameters until the best possible fit to the data is achieved. To prevent overfitting and ensure the model generalizes well to new, unseen data, a portion of the training dataset may be set aside as a validation set. This set may not be used in the primary training but serves as a benchmark to evaluate the model's performance and make necessary adjustments.

In some embodiments, the anomalies detection engine 124 may collaborate with the ML model 122 to identify one or more anomalies and/or abnormalities. For example, the anomalies detection engine 124 may receive a set of suckling data collected by the vacuum measurement device 102. The anomalies detection engine 124 may identify one or more abnormalities associated with the collected suckling data using the ML model 122. In some embodiments, the identified one or more abnormalities are corresponding to one or more of the plurality of dimensions. For example, the identified abnormalities may be corresponding with a frequency of the suckling, and this may provide a clinician with specificities and enhance clinician's diagnose.

In some embodiments, the visualization generation engine 126 may generate visualizations and/or graphic representations for one or more dataset. For example, the visualization generation engine 126 may generate a real-time visualization of the collected suckling data. In some embodiments, the visualization generation engine 126 may generate a real-time visualization of the collected suckling data in comparison to a population mean and standard deviation. In some embodiments, the visualization generation engine 126 may generate a graphical representation of the identified one or more abnormalities.

The data access modules 142 may facilitate access to data storage 150 of the server platform 120 by any of the remaining modules/engines 122, 124, and 126 of the server platform 120. In one example, one or more of the data access modules 142 may be database access modules, or may be any kind of data access module capable of storing data to, and/or retrieving data from, the data storage 150 according to the needs of the particular modules 122, 124, and 126 employing the data access modules 142 to access the data storage 150. Examples of the data storage 150 include, but are not limited to, one or more data storage components, such as magnetic disk drives, optical disk drives, solid state disk (SSD) drives, and other forms of nonvolatile and volatile memory components. The data storage 150 may store input clinical data and/or one or more determinations/models/digital twins made and/or generated by the remaining modules/engines 122, 124, and 126 of the server platform 120.

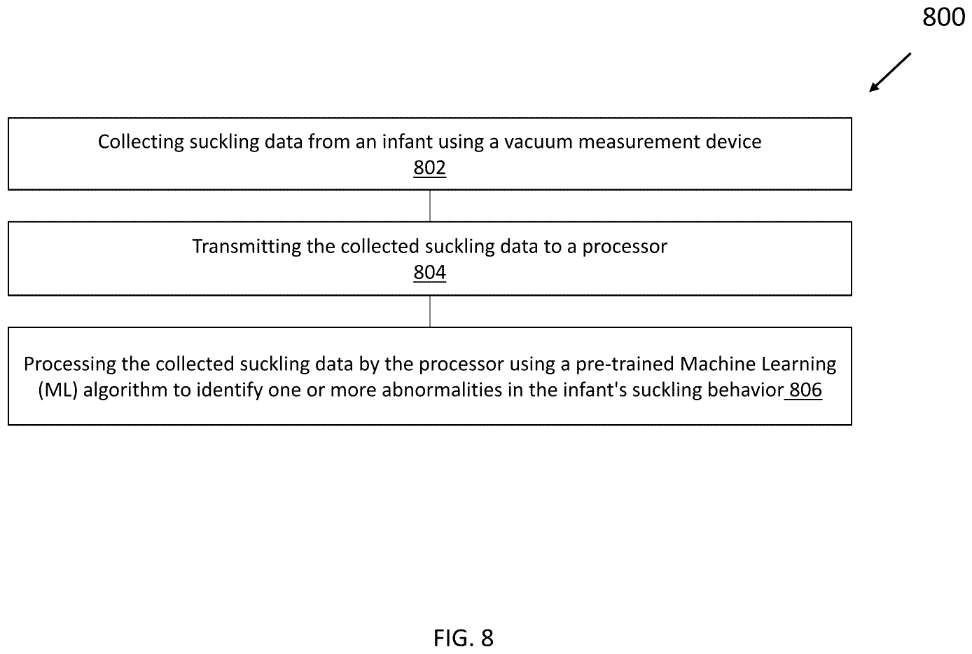

FIG. 8 depicts an example flowchart for a process 800 for evaluating infant suckling behavior, according to one or more embodiments of the present disclosure. As shown in FIG. 8, the process 800 may start with operating 802, wherein the system (e.g., system 700 as shown in FIG. 7) may collect suckling data from an infant using a vacuum measurement device (e.g., the vacuum measurement device 102 as shown in FIG. 7). In some embodiments, the vacuum measurement device 102 may automatically transmit the collected suckling data to a processor, as operation 804 shows. In some embodiments the vacuum measurement device 102 may transmit the collected suckling data to the processor in real-time or near real-time. The process may proceed to operation 806, wherein the processor may process the collected suckling data using a pre-trained Machine Learning (ML) algorithm (e.g., ML model 122 as shown in FIG. 7) to identify one or more abnormalities in the infant's suckling behavior. As discussed herein elsewhere, the ML model may be pre-trained using a set of training data, and the training data may comprise multiple dimensions. In some embodiments, the identified one or more abnormalities are corresponding to one or more of the plurality of dimensions. For example, the identified abnormalities may be corresponding with a frequency of the suckling, and this may provide a clinician with specificities and enhance clinician's diagnose.

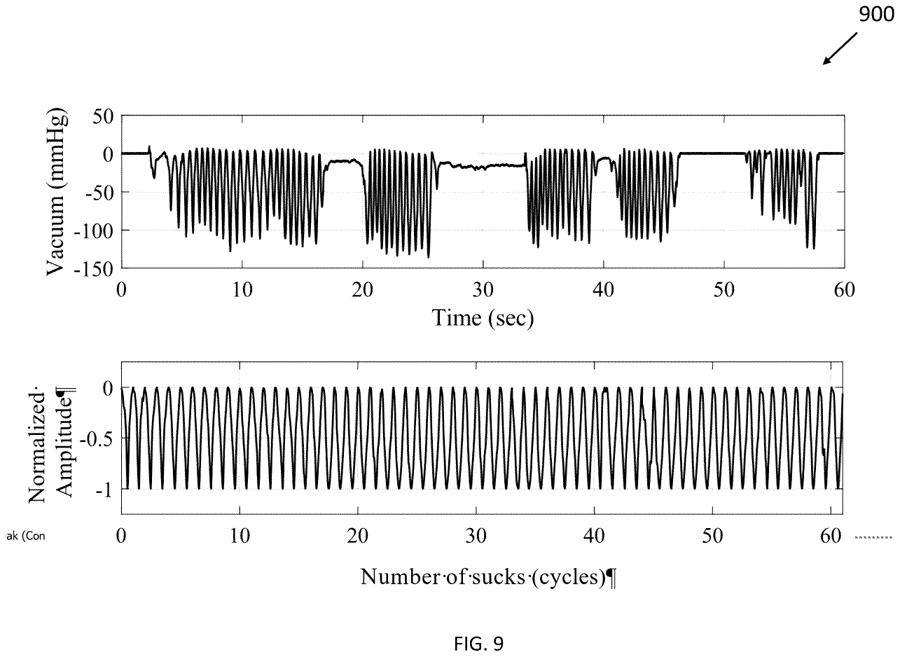

FIG. 9 depicts signals representative of a 60-second non-nutritive suckling shape as measured, according to one or more embodiments of the present disclosure. As depicted in the top panel of FIG. 9, the raw non-nutritive suckling data exhibits a degree of variability, reflecting the inherent unpredictability of infant suckling patterns. This data captures 61 distinct suckling events in this timeframe. The bottom panel of FIG. 9, however, illustrates the transformation achieved through normalization of the aforementioned raw data. By employing normalization techniques, each suckling event is restructured to produce a much more consistent and standardized signal. Despite this regularization, it's worth noting that the intrinsic characteristics of each suckling event are retained, ensuring that the data remains reflective of the original suckling pattern. Furthermore, the parameters in the normalized graph are dimensionless, making it easier to compare and analyze across various datasets.

In some embodiments, the non-nutritive suckling (NNS) vacuum of an infant is measured to derive characteristics of their suckling behavior. A specialized NNS device, in particular embodiments, captures the suckling vacuum over a span of sixty seconds, producing data that are indicative of the nuances of non-nutritive suckling. From a cohort of 91 subjects, parameters (i.e., dimensions of data set) such as mean suck vacuum, maximum suck vacuum, suckling frequency, burst duration, sucks per burst, and suckling shape are extracted for each individual. In some embodiments, methods and techniques have been detailed for obtaining parameters like mean suck vacuum, maximum suck vacuum, suckling frequency, burst duration, and sucks per burst from the infant NNS signals. Extending from that foundation, the current disclosure introduces an additional evaluation metric: the infant's suckling shape. This metric, in some embodiments, defines the visual representation of the vacuum in relation to time. It has been documented that a typical infant suckling pattern manifests as a smooth, regular, and almost sinusoidal curve. Deviations from this normative pattern may indicate potential irregularities in the infant's suckling mechanism, and such deviations can be detected and analyzed through frequency analysis techniques.

As discussed herein elsewhere, three distinctive infant suckling shapes have been identified: smooth sinusoidal, “sharp valley”, and “double valley”. These classifications provide insights into potential abnormalities within the signal that may correlate with disordered suckling. In some embodiments, it is observed that the amplitude and period of the NNS signals can exhibit variability throughout the monitoring phase. To ascertain the inherent characteristics of the suckling vacuum signal's shape over time, individual suckling events are isolated and normalized, both in amplitude (ranging from −1 to 0) and period (standardized to 0.5 sec). By this operational definition, a suckling cycle encompasses the peak-valley-peak profile observed during the suckling phase, as detailed in the disclosures. In order to dissect and categorize the prominent frequencies embedded within the normalized NNS signal, the data is subjected to a fast Fourier transform (FFT) in some embodiments. This analysis consistently emphasizes primary frequencies, especially at 4 Hz, 6 Hz, and 8 Hz, which recur in a majority of NNS signal data. Such frequency occurrences have been underscored in previous infant suckling measurements. In some embodiments, the amplitude signals generated at these specific frequencies are catalogued and are considered representative features of each infant's distinctive suckling shape.

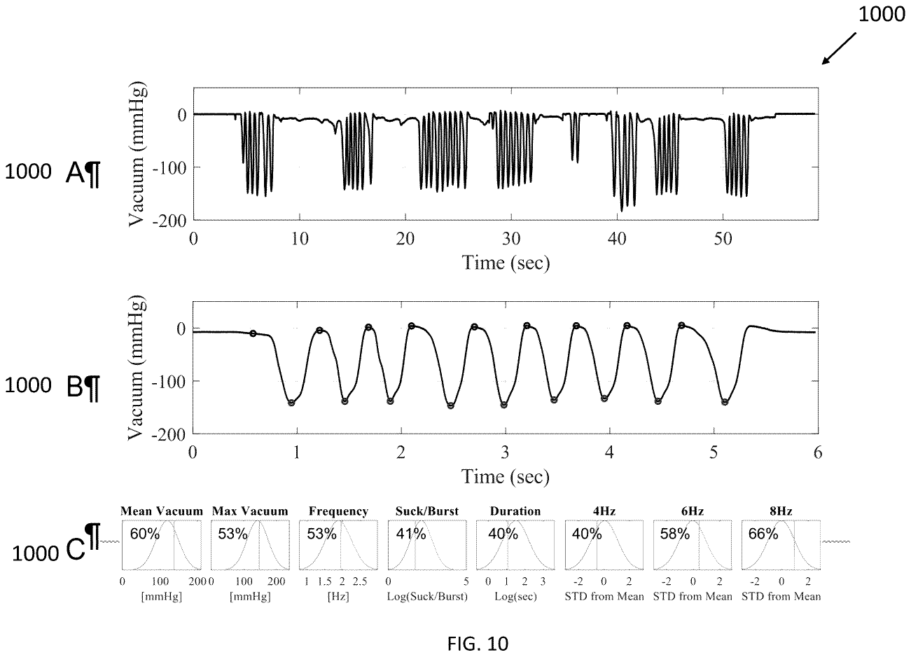

FIG. 10 depicts a visualization of a typical infant's NNS behavior, according to one or more embodiments of the present disclosure. As shown in FIG. 10, a detailed visualization of a typical infant's NNS behavior, along with the computational outcomes of the eight key parameters that define its primary attributes. As shown in FIG. 10, an expansive view 1000A showcasing the complete NNS activity captured over a span of 60 seconds. A magnified segment 1000B, illustrating a concise 6-second excerpt from the third distinct suckling burst within the full 60-second measurement. A series of plots 1000C provides a quantitative depiction of the distribution, frequency, and trends of each of the eight parameters derived from NNS data.

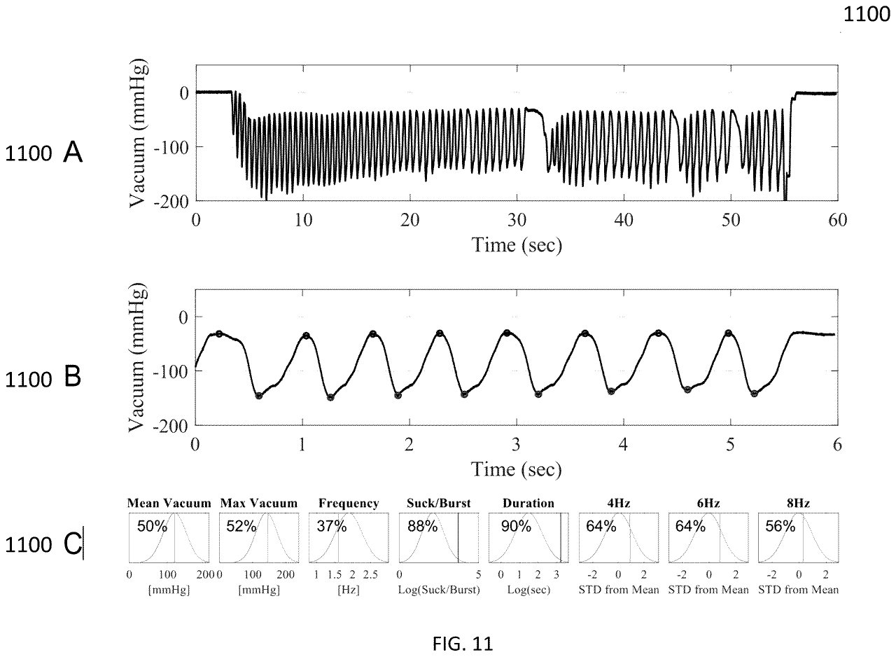

FIG. 11 depicts a visualization of the non-nutritive suckling (NNS) behavior of an infant characterized by prolonged bursts of suckling activity, according to one or more embodiments of the present disclosure. As shown in FIG. 11, a comprehensive display 1100A of the NNS pattern captured over a continuous 60-second interval is provided. As shown in FIG. 11, a zoomed-in view 1100B detailing a specific 6-second segment extracted from the initial extended suckling burst in the overall 60-second duration is provided. As shown in FIG. 11, A collection of graphs 1100C highlighting the statistical distribution, variations, and tendencies of the eight key parameters deduced from the NNS data of the said infant is provided.

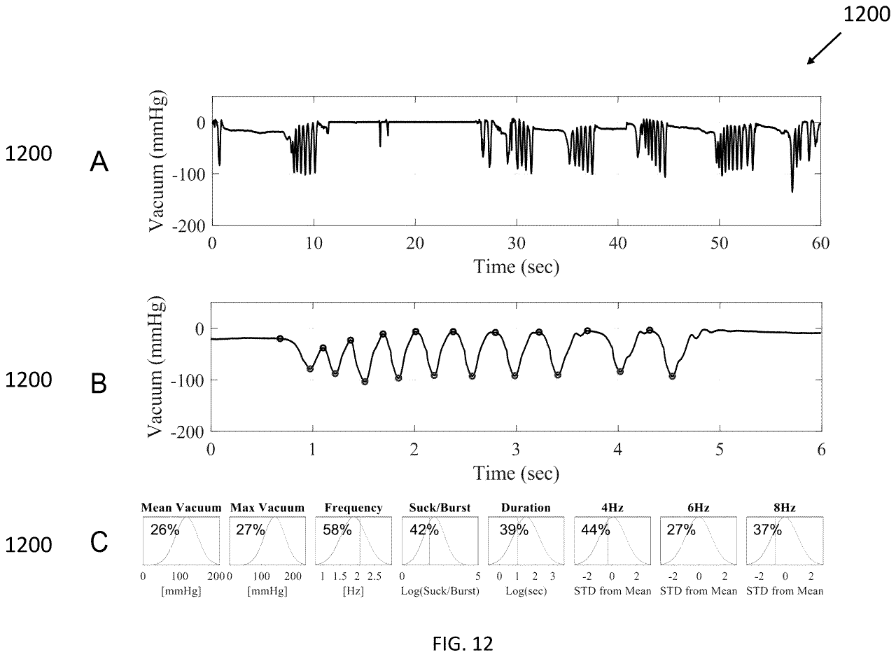

FIG. 12 depicts a visualization of the non-nutritive suckling (NNS) patterns of an infant who exhibits notably weak and sporadic suckling episodes, according to one or more embodiments of the present disclosure. As shown in FIG. 12, As shown in FIG. 12. An overarching visualization 1200A of the NNS behavior captured over an uninterrupted 60-second time frame is provided. As shown in FIG. 12, a focused snapshot 1200B capturing a specific 6-second segment extracted from the fifth suckling burst within the 60-second observation window is provided. As shown in FIG. 12, a series of graphical representations 1200C that delineate the statistical distribution, variations, and patterns of the eight primary parameters derived from the NNS data of the examined infant is provided.

In some embodiments, the objective is to determine whether a mere assessment of the suckling vacuum provides sufficient insight to detect potential breastfeeding difficulties. Furthermore, the potential utility of a set of parameters derived from this measurement is considered for characterizing an infant's feeding patterns. Three exemplary cases, both normal and aberrant, are showcased, derived from visual examinations of the suckling profiles and the distributions of the eight NNS parameters. These cases are contextualized within the broader dataset encompassing 91 infants.

Each illustrative case offers:
(a) An NNS recording spanning sixty seconds,
(b) A representative six-second extraction, and
(c) A statistical assessment of eight crucial parameters. It should be noted that the vertical indicators within the statistical visualizations denote the specific values associated with the respective case under examination.

In the depiction provided in FIG. 10, data from a healthy 12-day old infant is showcased, which demonstrates standard suckling behavior. The acquired measurements predominantly align within a single standard deviation from the mean values spanning the complete cohort. Additionally, the exhibited suckling pattern presents itself as rhythmic, closely mirroring a sinusoidal formation.

Contrastingly, FIG. 11 delineates measurements procured from a 6-day old infant. Notably, certain measurements deviate by roughly two standard deviations from the collective mean values. The suckling pattern, in this instance, conspicuously lacks regularity throughout the documented period. Distinctly, three pauses can be observed, of which two are exceptionally short-lived. Each individual suckling event manifests a pattern diverging from the standard sinusoidal waveform, collectively suggesting an anomalous suckling behavior. Upon clinical evaluation, this infant was found to have symptoms like hemorrhagic nipple lacerations, pronounced nipple discomfort, and episodes of choking, attributed to uncoordinated suck-swallow-breathe sequences. Such sustained, uninterrupted suckling, as discerned from the NNS data, may potentially underlie these clinical observations.

FIG. 12 portrays the suckling behavior of another infant, documented on the 18th day post-birth. While a majority of the measurements seem to fall within anticipated ranges, the suckling pattern is punctuated by brief active phases, interspaced with extended periods of inactivity. A closer examination of the fifth burst reveals certain inconsistencies towards its culmination. Notably, the mean and maximum suckling vacuum measurements were found to be positioned at the 26th and 27th percentiles, respectively. These observations align with clinical notes indicating a suboptimal latch. This particular infant was noted to be restless and exhibited symptoms of gastroesophageal reflux, a condition potentially linked with periodic detachment during feeding.

Conclusively, the provided illustrations underscore discernible differences between typical and atypical suckling behaviors as captured via NNS data. Subsequent sections will delve into NNS data stemming from multiple clinically identified instances of irregular feeding patterns

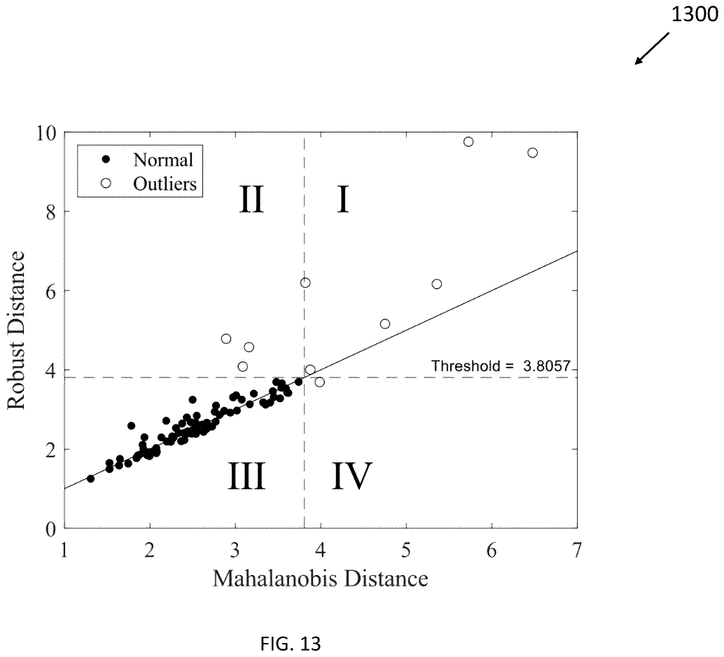

FIG. 13 illustrates a plot comparing the Robust distance and the Mahalanobis distance derived from the NNS data of all 91 subjects. Delineated on the plot are vertical and horizontal threshold lines (depicted as dashed) which were determined based on a value of 3.8 standard deviations. This value was grounded in the anticipated 7% outlier proportion and the dataset's 8 degrees of freedom. The intersecting threshold lines demarcate the plot into four distinct quadrants:
Quadrant III: This quadrant encompasses neonates that exhibit standard or normal NNS outcomes.
Quadrant II: Subjects located in this section are identified as outliers based solely on the Mahalanobis distance criterion.
Quadrant IV: Neonates positioned here are deemed outliers based on the Robust distance measurement.
Quadrant I: This quadrant comprises subjects that are flagged as outliers under both the Mahalanobis and Robust distance metrics.

The presented plot facilitates a comprehensive understanding of the distribution and classification of subjects based on the two distance metrics in the context of NNS data evaluation.

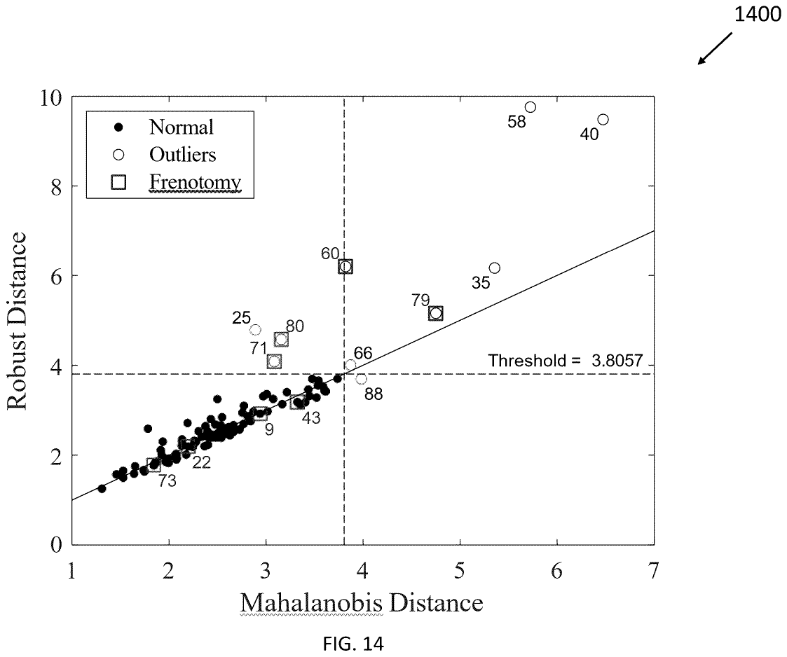

FIG. 14 illustrates presents a plot comparing the Robust distance and the Mahalanobis distance for the NNS data across the 91 infants. Among these infants, eight have been highlighted using red boxes, signifying that they underwent frenotomies. Within the context of the plot:
Four of these highlighted infants (marked with red boxes) are positioned as outliers based on the comparison of Robust distance and Mahalanobis distance metrics.
Conversely, the remaining four infants, also marked with red boxes, fall within the region representing the normal group in the plot.

This visual representation offers insight into the distribution of infants who underwent frenotomies, indicating a spread between both the outlier and normal classifications in the context of the NNS data evaluation.

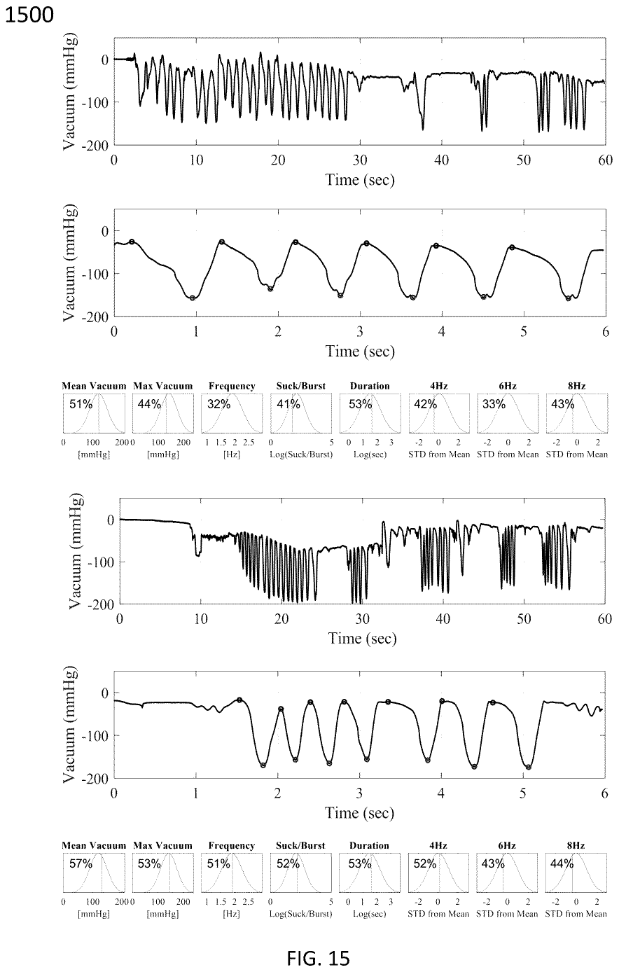

FIG. 15 provides a comprehensive visualization of the impact of a frenotomy on subject 71, specifically focusing on the non-nutritive suckling (NNS) parameters. The visualizations are categorized into two sets, each containing three plots:
The first set, denoted by plots (a), (b), and (c), displays the NNS data before the frenotomy. It encompasses the full 60-second measurement, a detailed 6-second segment, and a statistical assessment of the eight crucial parameters.
The subsequent set, highlighted by plots (d), (e), and (f), represents the NNS metrics following the frenotomy. Similar to the prior set, it illustrates the comprehensive 60-second data, a focused 6-second interval, and a statistical breakdown of the eight parameters.

From a comparative viewpoint, post-frenotomy observations, as showcased in the latter set, demonstrate a noticeable shift in both the frequency of suckling and the suck-per-burst metric, aligning them closer to the average values observed in the entire data set. The pre-frenotomy NNS data was captured on day 1 of the infant's life, with the frenotomy also being performed on that day. In contrast, the NNS metrics post-intervention were recorded on day 18, providing insight into the procedure's longitudinal effects.

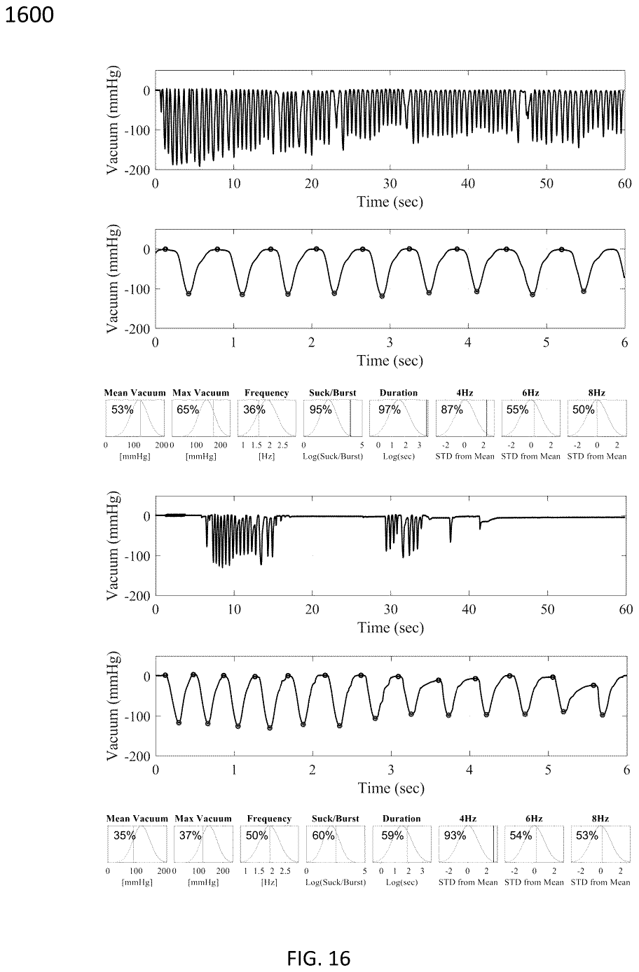

FIG. 16 provides the discernible influence of a frenotomy on subject 60, particularly emphasizing the non-nutritive suckling (NNS) metrics. The representations are split into two distinct sets, each encompassing three plots:
The initial set, denoted by plots (a), (b), and (c), presents the NNS data prior to the frenotomy. This set provides a comprehensive 60-second measurement, a detailed 6-second excerpt, and a statistical analysis of the eight vital parameters.
The subsequent set, as indicated by plots (d), (e), and (f), offers insights into the NNS metrics post-frenotomy. As with the previous set, it depicts the overall 60-second data, a specific 6-second snippet, and a statistical overview of the eight parameters.

In examining the post-frenotomy data, there's a shift in most of the parameters. Notably, all parameters—with the exception of the mean vacuum and the 4-Hz amplitude parameter—tended to gravitate toward the dataset's mean. These values were observed to fall within half a standard deviation of the mean after the procedure. Importantly, both the pre and post-frenotomy NNS data were captured on the first day of the infant's life, highlighting the immediate effects of the frenotomy within a short time span.

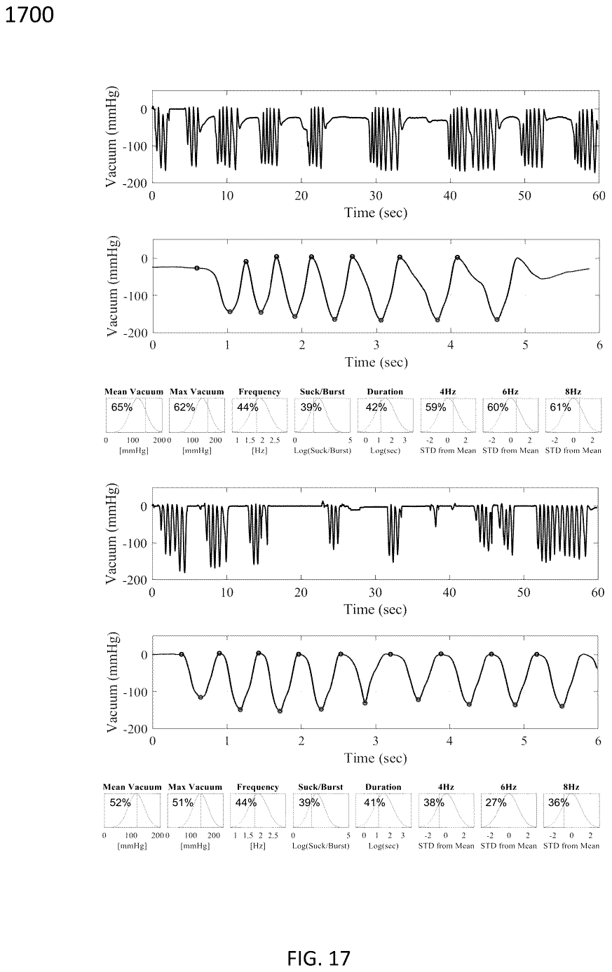

FIG. 17 presents a comparative analysis of subject 22's non-nutritive suckling (NNS) metrics pre and post-frenotomy. The displays are segmented into two principal sets:
The initial set, as represented by plots (b) and (c), captures the NNS metrics prior to the frenotomy. It comprises a detailed 6-second sample and a statistical assessment of the eight essential parameters.
The subsequent set, depicted by plots (e) and (f), reveals the NNS data following the frenotomy. Similarly, it offers a 6-second detailed glimpse and a statistical breakdown of the eight parameters.

Upon evaluating the post-frenotomy data, it's determined that the frenotomy had a limited impact on the subject's NNS parameters. A majority of the parameters, barring the 4, 6, and 8-Hz amplitude parameters, either remained consistent or demonstrated a marginal, statistically insignificant shift toward the mean. Conversely, the amplitude parameters at 4, 6, and 8-Hz frequencies notably deviated further from the dataset's mean subsequent to the frenotomy. This observation underscores the variability in frenotomy outcomes across different subjects.

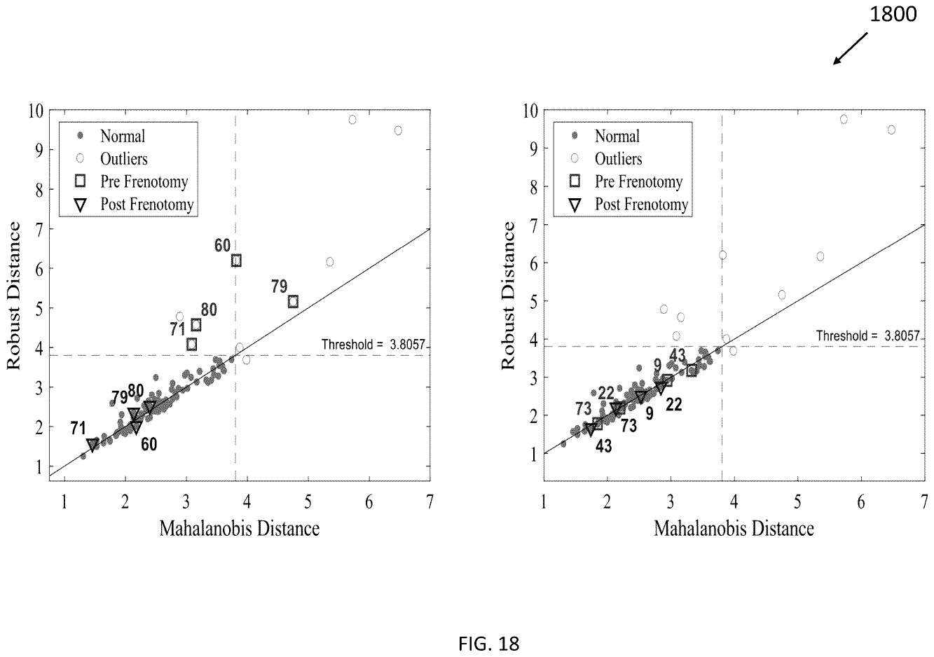

FIG. 18 provides a comparative visualization using the Robust distance versus Mahalanobis distance plot, specifically focusing on the NNS data from cases where a frenotomy was conducted. The intention is to differentiate the effects of the procedure-both before and after its execution.
Plot (a):
   Initial Condition: This segment marks the pre-frenotomy cases where NNS measurements indicated abnormal suckling behaviors. Such cases are delineated in red.
   Post-frenotomy Impact: Following the frenotomy procedure, the data indicates a restoration of suckling behavior towards the standard range. These improved cases are highlighted in blue.
Plot (b):
   Initial Condition: Here, cases that originally showcased normal NNS measurements are highlighted in red.
   Post-frenotomy Impact: Post the procedure, the NNS measurements largely remain consistent, indicating minimal changes in suckling behavior. These cases are symbolized in blue.

This visual representation underscores the multifaceted and occasionally contentious nature of the frenotomy procedure. It's evident that its impact varies among infants: while some showcase marked improvement post-procedure, others do not. By harnessing the capabilities of machine learning, it becomes feasible to pinpoint those infants that might benefit from the procedure with a higher degree of accuracy. Additionally, the efficacy of the frenotomy can be quantified in a more objective manner post-intervention.

As discussed herein elsewhere, in some embodiments, the NNS apparatus demonstrates the capability to detect infants exhibiting suckling irregularities potentially due to conditions like ankyloglossia and generalized infant vacuum suckling anomalies. NNS measurements indicate that for certain instances, the presence of lingual restrictions, which could lead to breastfeeding complications, may be effectively remedied through frenotomy, thereby normalizing infant suckling patterns. Notably, NNS data illustrates the nuanced nature of prevailing clinical practices surrounding ankyloglossia diagnoses. Some infants, despite presenting typical suckling mechanics via NNS analysis, still undergo frenotomies, yielding marginal alterations in their NNS readings.

In some embodiments, while various devices and methodologies might employ ultrasound, force sensors, sensor arrays, and cameras to enhance measurement accuracy, these can often introduce data complexity, user challenges, and elevated costs. In contrast, the presented embodiment emphasizes leveraging a singular metric—the suckling vacuum. Although other sensing techniques might be considered in the future, the depth of information derived from the suckling vacuum-time data suggests that extensive insights can be obtained from this lone parameter. In some embodiments, as discussed it is pivotal to provide clinicians with immediate access to measurement outcomes. Given the inherent challenges associated with employing technological apparatuses—particularly with restless infants in unfamiliar clinical settings—it becomes imperative that real-time data is available. Such immediate feedback empowers clinicians to discern potential anomalies, whether related to infant behavior (like refusal to suckle) or device malfunctions, and implement corrective measures promptly. This is especially advantageous during the initial days of an infant's life as milk production and breastfeeding habits solidify. Additionally or alternatively, results should be graphically presented in juxtaposition with population averages and standard deviations. By doing so, clinicians can swiftly pinpoint anomalies, providing a quantitative assessment that obviates the need for intricate data tables.

Illustrative cases in the results, as depicted in Figures, substantiate the utility of these principles for quantitative evaluation. This aids in determining the need for frenotomy and evaluating its effectiveness post-procedure. Further, unsupervised machine learning, employing robust and Mahalanobis distances, has been deployed to recognize anomalies. With a threshold calculated using a 7% outlier fraction corresponding to the clinical prevalence of ankyloglossia, ten out of the 91 infants exhibited outlier NNS data. Of these ten, four underwent frenotomies, subsequently showcasing enhanced NNS readings. Among the 81 infants with typical NNS outcomes, four underwent frenotomies, with varied post-operative results.

While the current study primarily targets anomalies stemming from ankyloglossia, optimizing the NNS data for diagnosing other oral dysfunctions necessitates compiling additional clinically correlated NNS data to discern and characterize these comparatively rarer oral shortcomings. Nevertheless, an integrated NNS-centric approach offers promise as a preliminary diagnostic tool in clinical settings. One inherent limitation across methodologies remains the continuous, long-term monitoring of infant suckling development. Infants' suckling behaviors evolve over time, potentially leading to enhanced, regressed, or consistent patterns that may not be fully captured within singular data points. Future endeavors should consider recurrent monitoring at various stages, especially post-interventions, to discern between intervention impacts and natural infant development. With expanded datasets and machine learning, variations in suckling maturation can be addressed more holistically. This research primarily emphasizes vacuum data, but anticipates the inclusion of expression pressure as an additional parameter in subsequent machine learning algorithms.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:

Example 1: A method for evaluating infant suckling behavior, comprising: collecting suckling data from an infant using a vacuum measurement device, transmitting the collected suckling data to at least one processor, and processing the collected suckling data by the at least one processor using a pre-trained Machine Learning (ML) algorithm to identify one or more abnormalities in the infant's suckling behavior, wherein the ML algorithm has been trained on a set of training data comprising a plurality of dimensions associated with the training data, and wherein the identified one or more abnormalities are corresponding to one or more of the plurality of dimensions.

Example 2: The method of Example 1, wherein the vacuum measurement device comprises a pacifier integrated with a feeding tube.

Example 3: The method of any of Examples 1-2, further comprising: providing, on a display, a real-time visualization of the collected suckling data.

Example 4: The method of any of Examples 1-3, further comprising, providing, on the display, a real-time visualization of the collected suckling data in comparison to a population mean and standard deviation.

Example 5: The method of any of Examples 1-4, further comprising: providing, on the display, a graphical representation of the identified one or more abnormalities.

Example 6: The method of any of the Examples 1-5: wherein the plurality of dimensions comprise mean suck vacuum, maximum suck vacuum, suckling frequency, burst duration, sucks per burst, and vacuum signal shape.

Example 7: The method of any of the Examples 1-6: wherein the ML algorithm is configured to apply an unsupervised anomaly detection method using the Mahalanobis distance to detect abnormalities in the collected suckling data.

Example 8: A system for evaluating infant suckling, the system comprising, a vacuum measurement device configured to collect suckling data from an infant; and at least one processor configured to receive the collected suckling data and process the collected suckling data using a pre-trained Machine Learning (ML) algorithm to identify one or more abnormalities in the infant's suckling behavior, wherein the ML algorithm has been trained on a set of training data comprising a plurality of dimensions associated with the training data, and wherein the identified one or more abnormalities are corresponding to one or more of the plurality of dimensions.

Example 9: The system of Example 8: wherein the vacuum measurement device comprises a pacifier integrated with a feeding tube.

Example 10: The system of any of Examples 8-9: wherein the vacuum measurement device comprises a pacifier integrated with a feeding tube. the vacuum measurement device comprises: a pacifier, a feeding tube, wherein a first end of the feeding tube is coupled with the pacifier, and a pressure sensor that is connected with a second end of the feeding tube, wherein the pressure sensor is configured to collect the suckling data from the infant.

Example 11: The system of any of the Examples 8-10: wherein the system further comprises a display configured to provide real-time visualization of the collected suckling data.

Example 12: The system of any of the Examples 8-11: wherein the display is further configured to provide a real-time visualization of the collected suckling data in comparison to a population mean and standard deviation.

Example 13: The system of any of the Examples 8-12: wherein the display is further configured to provide a real-time visualization of the collected suckling data in comparison to a population mean and standard deviation.

Example 14: The system of any of the Examples 8-13: wherein the plurality of dimensions comprise mean suck vacuum, maximum suck vacuum, suckling frequency, burst duration, sucks per burst, and vacuum signal shape.

Example 15: The system of any of the Examples 8-14: wherein the ML algorithm is configured to apply an unsupervised anomaly detection method using the Mahalanobis distance to detect abnormalities in the collected suckling data.

Example 16: A vacuum measurement device, comprising: a pacifier, a feeding tube, wherein a first end of the feeding tube is coupled with the pacifier, and a pressure sensor that is connected with a second end of the feeding tube, wherein the pressure sensor is configured to collect suckling data from an infant.

Example 17: the vacuum measurement device of the Example 16, wherein the feeding tube is shorter than 48 cm.

Example 18: the vacuum measurement device of any of the Examples 16-17, further comprising a transmission unit configured to transmit the collected sucking data to at least one processor.

Example 19: the vacuum measurement device of any of the Examples 16-18, wherein the pressure sensor is configured to detect and measure changes in pressure within the pacifier as the infant suckles.

Example 20: the vacuum measurement device of any of the Examples 16-19, wherein the pressure sensor is configured to detect and measure a plurality of dimensions comprising mean suck vacuum, maximum suck vacuum, suckling frequency, burst duration, sucks per burst, and vacuum signal shape.

Computer programs, which can also be referred to programs, software, software applications, applications, components, or code, may be used and include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user can provide input to the computer. Other kinds of devices can be used to provide for interaction with a user as well. For example, feedback provided to the user can be any form of sensory feedback, such as for example visual feedback, auditory feedback, or tactile feedback; and input from the user can be received in any form, including, but not limited to, acoustic, speech, or tactile input.

The subject matter described herein can be implemented in a computing system that includes a back-end component, such as for example one or more data servers, or that includes a middleware component, such as for example one or more application servers, or that includes a front-end component, such as for example one or more client computers having a graphical user interface or a Web browser through which a user can interact with an implementation of the subject matter described herein, or any combination of such back-end, middleware, or front-end components. The components of the system can be interconnected by any form or medium of digital data communication, such as for example a communication network. Examples of communication networks include, but are not limited to, a local area network (“LAN”), a wide area network (“WAN”), and the Internet.

The computing system can include clients and servers. A client and server are generally, but not exclusively, remote from each other and typically interact through a communication network. The relationship of client and server arises by virtue of computer programs running on the respective computers and having a client-server relationship to each other.

Although ordinal numbers such as first, second, and the like can, in some situations, relate to an order; as used in this document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations can be within the scope of the following claims.

What is claimed:

1. A method for evaluating infant suckling behavior, comprising:
collecting suckling data from an infant using a vacuum measurement device,
transmitting the collected suckling data to at least one processor, and
processing the collected suckling data by the at least one processor using a pre-trained Machine Learning (ML) algorithm to identify one or more abnormalities in the infant's suckling behavior, wherein the ML algorithm has been trained on a set of training data comprising a plurality of dimensions associated with the training data, and wherein the identified one or more abnormalities are corresponding to one or more of the plurality of dimensions.

2. The method of claim 1, wherein the vacuum measurement device comprises a pacifier integrated with a feeding tube.

3. The method of claim 1, further comprising:
providing, on a display, a real-time visualization of the collected suckling data.

4. The method of claim 3, further comprising,
providing, on the display, a real-time visualization of the collected suckling data in comparison to a population mean and standard deviation.

5. The method of claim 3, further comprising:
providing, on the display, a graphical representation of the identified one or more abnormalities.

6. The method of claim 1, wherein the plurality of dimensions comprise mean suck vacuum, maximum suck vacuum, suckling frequency, burst duration, sucks per burst, and vacuum signal shape.

7. The method of claim 1, wherein the ML algorithm is configured to apply an unsupervised anomaly detection method using the Mahalanobis distance to detect abnormalities in the collected suckling data.

8. A system for evaluating infant suckling, the system comprising,
a vacuum measurement device configured to collect suckling data from an infant; and
at least one processor configured to receive the collected suckling data and process the collected suckling data using a pre-trained Machine Learning (ML) algorithm to identify one or more abnormalities in the infant's suckling behavior, wherein the ML algorithm has been trained on a set of training data comprising a plurality of dimensions associated with the training data, and wherein the identified one or more abnormalities are corresponding to one or more of the plurality of dimensions.

9. The system of claim 8, wherein the vacuum measurement device comprises a pacifier integrated with a feeding tube.

10. The system of claim 8, wherein the vacuum measurement device comprises:
a pacifier,
a feeding tube, wherein a first end of the feeding tube is coupled with the pacifier, and
a pressure sensor that is connected with a second end of the feeding tube, wherein the pressure sensor is configured to collect the suckling data from the infant.

11. The system of claim 8, further comprising:
a display configured to provide real-time visualization of the collected suckling data.

12. The system of claim 11, wherein the display is further configured to provide a real-time visualization of the collected suckling data in comparison to a population mean and standard deviation.

13. The system of claim 11, wherein the display is further configured to provide a graphical representation of the identified one or more abnormalities.

14. The system of claim 8, the plurality of dimensions comprise mean suck vacuum, maximum suck vacuum, suckling frequency, burst duration, sucks per burst, and vacuum signal shape.

15. The system of claim 8, wherein the ML algorithm is configured to apply an unsupervised anomaly detection method using the Mahalanobis distance to detect abnormalities in the collected suckling data.

16. A vacuum measurement device, comprising:
a pacifier,
a feeding tube, wherein a first end of the feeding tube is coupled with the pacifier, and
a pressure sensor that is connected with a second end of the feeding tube, wherein the pressure sensor is configured to collect suckling data from an infant.

17. The vacuum measurement device of claim 16, wherein the feeding tube is shorter than 48 cm.

18. The vacuum measurement device of claim 16, further comprising:
a transmission unit configured to transmit the collected sucking data to at least one processor.

19. The vacuum measurement device of claim 16, wherein the pressure sensor is configured to detect and measure changes in pressure within the pacifier as the infant suckles.

20. The vacuum measurement device of claim 16, wherein the pressure sensor is configured to detect and measure a plurality of dimensions comprising mean suck vacuum, maximum suck vacuum, suckling frequency, burst duration, sucks per burst, and vacuum signal shape.
